(19) United States
(12) Patent Application Publication
(10) Pub. No.: US 2016/0166773 A1
(43) Pub. Date: Jun. 16, 2016

(54) INDICATOR AND DELIVERY DEVICE

(71) Applicant: SANOFI, Paris (FR)

(72) Inventors: Tom Oakley, Cambridge Cambridgeshire (GB); Matt Schumann, Bourn, Cambridge, Cambridgeshire (GB); Stuart Milne, Buckden St. Neots, Cambridgeshire (GB)

(21) Appl. No.: 14/905,153

(22) PCT Filed: Jul. 17, 2014
(86) PCT No.: PCT/EP2014/065331

§ 371 Date: Jan. 14, 2016

(30) Foreign Application Priority Data
Jul. 17, 2013 (EP) 13176854.1

(51) Int. Cl. A61M 5/315

(52) U.S. Cl. CPC A61M 5/31525; A61M 2005/3126

(57) ABSTRACT

An indicator for a delivery device comprising a belt (71) having marks (73) for dose indication and/or status indication.

The invention concerns an indicator for a delivery device and such a delivery device, in particular a drug delivery device.

In an exemplary delivery device, a bung within a cartridge that contains a liquid or a paste may be displaced by a piston rod, thereby delivering a dose. The delivery device comprises a drive mechanism which allows setting and delivering the dose by means of piston rod movement. A drug delivery device is a delivery device in the medical field. The drug delivery device may be embodied as a pen injector suitable for delivering several doses of a drug. Document EP 1322355 shows a device for carrying out the dosed administration of an injectable product. Document WO 2009/095129 shows an injector with a thumb-operable scroll wheel. Document WO 2006/079481 shows an injection device with an end of dose feedback mechanism. Document WO 2010/046394 shows a dial-down mechanism for a wind-up pen. Document EP 1855742 shows a dosing device for setting fine doses. Documents U.S. Pat. No. 5,383,865 and U.S. Pat. No. 7,699,815 show medication delivery devices.

Many pen injectors have a dose indicator which displays the set dose to the user, and counts down to zero as the dose is dispensed. Pen injectors may also feature the rotation of at least one component to set the dose. The dose may be delivered by converting the rotational movement of setting the dose into translation movement of the bung in the cartridge. Therefore pen injectors may feature a number sleeve with dose indications on it which moves on a helix relative to the housing. The problems with running the number sleeve on a helix relative to the housing include the following aspects.

The number sleeve moves axially, which can make the device longer. The minimum axial movement is limited due to requirements on the minimum text size and the maximum number of doses which must be supported.

Devices with a large number of doses will require large axial displacements of the number sleeve, and it is difficult to prevent numbers being shown outside the dose indicator window, for example on the barrel of the number sleeve.

The geometry of the marks or text for indicating information is tied to the geometry of the drive mechanism. This can limit text size or require large displacements for dose setting and injection.

It is an aim of the invention to provide an alternative indicator suitable for indicating the set dose.

An indicator for a delivery device is provided, the dose indicator comprising a belt having marks for dose indication and/or status indication of the delivery device. Such a dose indicator may be used in a drug delivery device.

The marks may include visual or tactile marks indicating information about the operation status of the device, e.g. whether it is empty or whether it has been primed. Preferably the marks may indicate the set dose by means of symbols or digits.

A key aspect of the belt concept is that the indicator having a belt with marks, which may be numbers indicating the set dose, is not dependent on a rigid or semi-rigid number sleeve. Therefore many of the problems above are avoided.

Another advantage of the dose indicator including a belt is that the flexible nature of the belt element allows the dose indicator mechanism to be arranged in a wider range of form factors. In many cases this will allow a more efficient use of space within the device packaging. The indicator having a belt can be used in any device which would benefit from a non-axis-symmetric device format.

Additionally the dose number may be presented on a much flatter surface than a cylinder. This allows larger perceived dose numbers and less distortion when viewing at an angle. Should the device require additional magnification, a flatter display will allow greater freedom with the lens design and potentially less optical distortion. The indicator having a belt can be used in any device which where a clearer dosage display is desirable.

In one embodiment the belt runs over a first wheel means and a second wheel means; the belt, the first and second wheel means forming a belt gear suitable for transferring rotation from one of the wheel means to the other wheel means.

A belt is a loop of flexible material used to link the wheel means mechanically. A wheel means is preferably coupled with a shaft in such a manner that the rotation of the shaft rotates the wheel means. Thus the belt gear allows transmission of rotation from one shaft to another. A wheel means may be a region of the shaft or a wheel-shaped means on the shaft the belt runs over. The wheel means does not necessarily protrude from the shaft. The wheel means and the shaft may be integrally formed in one piece.

In one embodiment the belt is formed as toothed belt comprising teeth on its inner side, the teeth engaging with teeth on at least one of the first and second wheel means which may be toothed wheels or toothed regions of a shaft. When correctly tensioned, toothed belts have no slippage, run at constant speed, and are often used to transfer direct motion for indexing purposes.

In one embodiment, at least one of the first and second wheel means comprises a pulley over which a flat belt, i.e. a belt having no teeth and a rectangular, trapezoid or ellipsoid cross-section, runs. A flat belt may suitable for forming a simple system of power transmission.

The dose indicator is not limited to a single belt. It further comprises a further belt having marks for dose indication and/or status indication. More than two belts can be used. Increasing the number of belts allows the display of a greater amount of information.

In one embodiment the belt and the further belt are arranged adjacent and substantially parallel to each other. The marks on the belt include digits that indicate units of an amount of a set dose and the marks on the further belt including digits that indicate tens of units of the amount of the set dose. In this arrangement the belts form a counter for dosage indication. The belt may be coupled with the further belt in such a manner that the further belt is moved one increment when the belt completes one revolution.

The components may be coupled directly, e.g. being connected or engaged, or indirectly, i.e. by means of other components. Components that are coupled may or may not be able to move, e.g. axially and/or rotationally, with respect to each other.

One embodiment of the indicator comprises an escapement mechanism which features the stepwise movement of the further belt. Such an escapement mechanism couples the belt and the further belt in such a manner that the further belt moves by one increment, i.e. from one digit to the following digit, after one revolution of the belt.

The indicator may be used in delivery device, preferably a drug delivery device that further comprises a moveable dosage selector coupled by a shaft with the first wheel means in such a manner that a movement of the dosage selector causes rotation of the first wheel means. Such a dosage selector may be a scroll wheel that allows comfortable and easy dose setting.

The second wheel means may be coupled with a mechanism for driving a piston rod, the mechanism having one component that is moved by the second wheel means. In other words, the belt gear has a double function: on the one hand it provides dosage or status information; on the other hand it travels a component of the drive mechanism during dose setting, thereby adjusting the amount of the drug that is delivered by movement of the piston rod after dose setting.

The term “piston rod” shall preferably mean a component adapted to operate through/within a housing of the delivery device, which may be designed to move axially through/within the delivery device preferably from the follower member to the piston rod, for example for the purpose of discharging or dispensing an injectable product. “Piston rod” shall further mean a component having a circular or non-circular cross-section. It may be made of any suitable material known by a person skilled in the art and may be of unitary or multipart construction.

The housing of the delivery device may have an opening, a part of the belt being visible through the opening, wherein the visible mark indicates the set dose or status. The symbols in the window indicate the dose set or other information.

The opening may be covered by a translucent or transparent material. Such a cover may prevent debris from entering the opening and either looking unsightly or interfering with the operation of the dose indicator mechanism.

The opening may be covered by a lens. The cover could be shaped to affect a lens and therefore increase the apparent size of text, numbers or other information on the belts.

The delivery device may comprise an elastic member which is suitable for storing elastic energy for supporting the drug delivery. The elastic member may be a compression spring. Alternatively the elastic member may be a torsion spring.

The terms “drug” or “medicament”, as used herein, preferably mean a pharmaceutical formulation containing at least one pharmaceutically active compound,

wherein in one embodiment the pharmaceutically active compound has a molecular weight up to 1500 Da and/or is a peptide, a protein, a polysaccharide, a vaccine, a DNA, a RNA, an enzyme, an antibody or a fragment thereof, a hormone or an oligonucleotide, or a mixture of the above-mentioned pharmaceutically active compound,
wherein in a further embodiment the pharmaceutically active compound is useful for the treatment and/or prophylaxis of diabetes mellitus or complications associated with diabetes mellitus such as diabetic retinopathy, thromboembolism disorders such as deep vein or pulmonary thromboembolism, acute coronary syndrome (ACS), angina, myocardial infarction, cancer, macular degeneration, inflammation, hay fever, atherosclerosis and/or rheumatoid arthritis,
wherein in a further embodiment the pharmaceutically active compound comprises at least one peptide for the treatment and/or prophylaxis of diabetes mellitus or complications associated with diabetes mellitus such as diabetic retinopathy,
wherein in a further embodiment the pharmaceutically active compound comprises at least one human insulin or a human insulin analogue or derivative, glucagon-like peptide (GLP-1) or an analogue or derivative thereof, or exendin-3 or exendin-4 or an analogue or derivative of exendin-3 or exendin-4.

Insulin analogues are for example Gly(A21), Arg(B31), Arg(B32) human insulin; Lys(B3), Glu(B29) human insulin; Lys(B28), Pro(B29) human insulin; Asp(B28) human insulin; human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Insulin derivatives are for example B29-N-myristoyl-des(B30) human insulin; B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N—(N-palmitoyl-Y-glutamyl)-des(B30) human insulin; B29-N—(N-lithocholyl-Y-glutamyl)-des(B30) human insulin; B29-N-(ω-carboxyheptadecanoyl)-des(B30) human insulin and B29-N-(ω-carboxyheptadecanoyl) human insulin.

Exendin-4 for example means Exendin-4(1-39), a peptide of the sequence H-His-Gly-Glu-Gly-Thr-Phe-Thr-Ser-Asp-Leu-Ser-Lys-Gln-Met-Glu-Glu-Glu-Ala-Val-Arg-Leu-Phe-Ile-Glu-Trp-Leu-Lys-Asn-Gly-Gly-Pro-Ser-Ser-Gly-Ala-Pro-Pro-Pro-Ser-NH2.

Exendin-4 derivatives are for example selected from the following list of compounds:

### H-(Lys)4-des Pro36, des Pro37 Exendin-4(1-39)-NH2,

### H-(Lys)5-des Pro36, des Pro37 Exendin-4(1-39)-NH2,

### des Pro36 Exendin-4(1-39),

### des Pro36 [Asp28] Exendin-4(1-39),

### des Pro36 [IsoAsp28] Exendin-4(1-39),

### des Pro36 [Met(O)14, Asp28] Exendin-4(1-39),

### des Pro36 [Met(O)14, IsoAsp28] Exendin-4(1-39),

### des Pro36 [Trp(O2)25, Asp28] Exendin-4(1-39),

### des Pro36 [Trp(O2)25, IsoAsp28] Exendin-4(1-39),

### des Pro36 [Met(O)14 Trp(O2)25, Asp28] Exendin-4(1-39),

### des Pro36 [Met(O)14 Trp(O2)25, IsoAsp28] Exendin-4(1-39); or

### des Pro36 [Asp28] Exendin-4(1-39),

### des Pro36 [IsoAsp28] Exendin-4(1-39),

### des Pro36 [Met(O)14, Asp28] Exendin-4(1-39),

### des Pro36 [Met(O)14, IsoAsp28] Exendin-4(1-39),

### des Pro36 [Trp(O2)25, Asp28] Exendin-4(1-39),

### des Pro36 [Trp(O2)25, IsoAsp28] Exendin-4(1-39),

### des Pro36 [Met(O)14 Trp(O2)25, Asp28] Exendin-4(1-39),

### des Pro36 [Met(O)14 Trp(O2)25, IsoAsp28] Exendin-4(1-39),

wherein the group -Lys6-NH2 may be bound to the C-terminus of the Exendin-4 derivative;
or an Exendin-4 derivative of the sequence

### Des Pro36 Exendin-4(1-39)-Lys6-NH2 (AVE0010),

### H-(Lys)6-des Pro36 [Asp28] Exendin-4(1-39)-Lys6-NH2,

### des Asp28 Pro36, Pro37, Pro38Exendin-4(1-39)-NH2,

### H-(Lys)6-des Pro36, Pro38 [Asp28] Exendin-4(1-39)-NH2,

### H-Asn-(Glu)5des Pro36, Pro37, Pro38 [Asp28] Exendin-4(1-39)-NH2,

### des Pro36, Pro37, Pro38 [Asp28] Exendin-4(1-39)-(Lys)6-NH2,

### H-(Lys)6-des Pro36, Pro37, Pro38 [Asp28] Exendin-4(1-39)-(Lys)6-NH2,

### H-Asn-(Glu)5-des Pro36, Pro37, Pro38 [Asp28] Exendin-4(1-39)-(Lys)6-NH2,

### H-(Lys)6-des Pro36 [Trp(O2)25, Asp28] Exendin-4(1-39)-Lys6-NH2,

### H-des Asp28 Pro36, Pro37, Pro38 [Trp(02)25] Exendin-4(1-39)-NH2,

### H-(Lys)6-des Pro36, Pro37, Pro38 [Trp(O2)25, Asp28] Exendin-4(1-39)-NH2,

### H-Asn-(Glu)5-des Pro36, Pro37, Pro38 [Trp(O2)25, Asp28] Exendin-4(1-39)-NH2,

### des Pro36, Pro37, Pro38 [Trp(O2)25, Asp28] Exendin-4(1-39)-(Lys)6-NH2,

### H-(Lys)6-des Pro36, Pro37, Pro38 [Trp(O2)25, Asp28] Exendin-4(1-39)-(Lys)6-NH2,

### H-Asn-(Glu)5-des Pro36, Pro37, Pro38 [Trp(O2)25, Asp28] Exendin-4(1-39)-(Lys)6-NH2,

### H-(Lys)6-des Pro36 [Met(O)14, Asp28] Exendin-4(1-39)-Lys6-NH2,

### des Met(O)14 Asp28 Pro36, Pro37, Pro38 Exendin-4(1-39)-NH2,

### H-(Lys)6-desPro36, Pro37, Pro38 [Met(O)14, Asp28] Exendin-4(1-39)-NH2,

### H-Asn-(Glu)5-des Pro36, Pro37, Pro38 [Met(O)14, Asp28] Exendin-4(1-39)-NH2,

### des Pro36, Pro37, Pro38 [Met(O)14, Asp28] Exendin-4(1-39)-(Lys)6-NH2,

### H-(Lys)6-des Pro36, Pro37, Pro38 [Met(O)14, Asp28] Exendin-4(1-39)-(Lys)6-NH2,

### H-Asn-(Glu)5 des Pro36, Pro37, Pro38 [Met(O)14, Asp28] Exendin-4(1-39)-(Lys)6-NH2,

### H-Lys6-des Pro36 [Met(O)14, Trp(O2)25, Asp28] Exendin-4(1-39)-Lys6-NH2,

### H-des Asp28 Pro36, Pro37, Pro38 [Met(O)14, Trp(02)25] Exendin-4(1-39)-NH2,

### H-(Lys)6-des Pro36, Pro37, Pro38 [Met(O)14, Asp28] Exendin-4(1-39)-NH2,

### H-Asn-(Glu)5-des Pro36, Pro37, Pro38 [Met(O)14, Trp(O2)25, Asp28] Exendin-4(1-39)-NH2,

### des Pro36, Pro37, Pro38 [Met(O)14, Trp(O2)25, Asp28] Exendin-4(1-39)-(Lys)6-NH2,

### H-(Lys)6-des Pro36, Pro37, Pro38 [Met(O)14, Trp(O2)25, Asp28] Exendin-4(S1-39)-(Lys)6-NH2,

### H-Asn-(Glu)5-des Pro36, Pro37, Pro38 [Met(O)14, Trp(O2)25, Asp28] Exendin-4(1-39)-(Lys)6-NH2;

or a pharmaceutically acceptable salt or solvate of any one of the afore-mentioned Exendin-4 derivative.

Hormones are for example hypophysis hormones or hypothalamus hormones or regulatory active peptides and their antagonists as listed in Rote Liste, ed. 2008, Chapter 50, such as Gonadotropin (Follitropin, Lutropin, Choriongonadotropin, Menotropin), Somatropin (Somatropin), Desmopressin, Terlipressin, Gonadorelin, Triptorelin, Leuprorelin, Buserelin, Nafarelin, Goserelin.

A polysaccharide is for example a glucosaminoglycan, a hyaluronic acid, a heparin, a low molecular weight heparin or an ultra low molecular weight heparin or a derivative thereof, or a sulphated, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium.

Antibodies are globular plasma proteins (˜150 kDa) that are also known as immunoglobulins which share a basic structure. As they have sugar chains added to amino acid residues, they are glycoproteins. The basic functional unit of each antibody is an immunoglobulin (Ig)monomer (containing only one Ig unit); secreted antibodies can also be dimeric with two Ig units as with IgA, tetrameric with four Ig units like teleost fish IgM, or pentameric with five Ig units, like mammalian IgM.

The Ig monomer is a “Y”-shaped molecule that consists of four polypeptide chains; two identical heavy chains and two identical light chains connected by disulfide bonds between cysteine residues. Each heavy chain is about 440 amino acids long; each light chain is about 220 amino acids long. Heavy and light chains each contain intrachain disulfide bonds which stabilize their folding. Each chain is composed of structural domains called Ig domains. These domains contain about 70-110 amino acids and are classified into different categories (for example, variable or V, and constant or C) according to their size and function. They have a characteristic immunoglobulin fold in which two β sheets create a “sandwich” shape, held together by interactions between conserved cysteines and other charged amino acids.

There are five types of mammalian Ig heavy chain denoted by α, δ, ε, γ, and μ. The type of heavy chain present defines the isotype of antibody; these chains are found in IgA, IgD, IgE, IgG, and IgM antibodies, respectively.

Distinct heavy chains differ in size and composition; α and γ contain approximately 450 amino acids and δ approximately 500 amino acids, while μ and ε have approximately 550 amino acids. Each heavy chain has two regions, the constant region (C_{H}) and the variable region (V_{H}). In one species, the constant region is essentially identical in all antibodies of the same isotype, but differs in antibodies of different isotypes. Heavy chains γ, α and δ have a constant region composed of three tandem Ig domains, and a hinge region for added flexibility; heavy chains μ and ε have a constant region composed of four immunoglobulin domains. The variable region of the heavy chain differs in antibodies produced by different B cells, but is the same for all antibodies produced by a single B cell or B cell clone. The variable region of each heavy chain is approximately 110 amino acids long and is composed of a single Ig domain.

In mammals, there are two types of immunoglobulin light chain denoted by λ and κ. A light chain has two successive domains: one constant domain (CL) and one variable domain (VL). The approximate length of a light chain is 211 to 217 amino acids. Each antibody contains two light chains that are always identical; only one type of light chain, κ or λ, is present per antibody in mammals.

Although the general structure of all antibodies is very similar, the unique property of a given antibody is determined by the variable (V) regions, as detailed above. More specifically, variable loops, three each the light (VL) and three on the heavy (VH) chain, are responsible for binding to the antigen, i.e. for its antigen specificity. These loops are referred to as the Complementarity Determining Regions (CDRs). Because CDRs from both VH and VL domains contribute to the antigen-binding site, it is the combination of the heavy and the light chains, and not either alone, that determines the final antigen specificity.

An “antibody fragment” contains at least one antigen binding fragment as defined above, and exhibits essentially the same function and specificity as the complete antibody of which the fragment is derived from. Limited proteolytic digestion with papain cleaves the Ig prototype into three fragments. Two identical amino terminal fragments, each containing one entire L chain and about half an H chain, are the antigen binding fragments (Fab). The third fragment, similar in size but containing the carboxyl terminal half of both heavy chains with their interchain disulfide bond, is the crystalizable fragment (Fc). The Fc contains carbohydrates, complement-binding, and FcR-binding sites. Limited pepsin digestion yields a single F(ab′)2 fragment containing both Fab pieces and the hinge region, including the H—H interchain disulfide bond. F(ab′)2 is divalent for antigen binding. The disulfide bond of F(ab′)2 may be cleaved in order to obtain Fab′. Moreover, the variable regions of the heavy and light chains can be fused together to form a single chain variable fragment (scFv).

Pharmaceutically acceptable salts are for example acid addition salts and basic salts. Acid addition salts are e.g. HCl or HBr salts. Basic salts are e.g. salts having a cation selected from alkali or alkaline, e.g. Na+, or K+, or Ca2+, or an ammonium ion N+(R1)(R2)(R3)(R4), wherein R1 to R4 independently of each other mean: hydrogen, an optionally substituted C1-C6-alkyl group, an optionally substituted C2-C6-alkenyl group, an optionally substituted C6-C10-aryl group, or an optionally substituted C6-C10-heteroaryl group. Further examples of pharmaceutically acceptable salts are described in “Remington's Pharmaceutical Sciences” 17. ed. Alfonso R. Gennaro (Ed.), Mark Publishing Company, Easton, Pa., U.S.A., 1985 and in Encyclopedia of Pharmaceutical Technology.

Pharmaceutically acceptable solvates are for example hydrates.

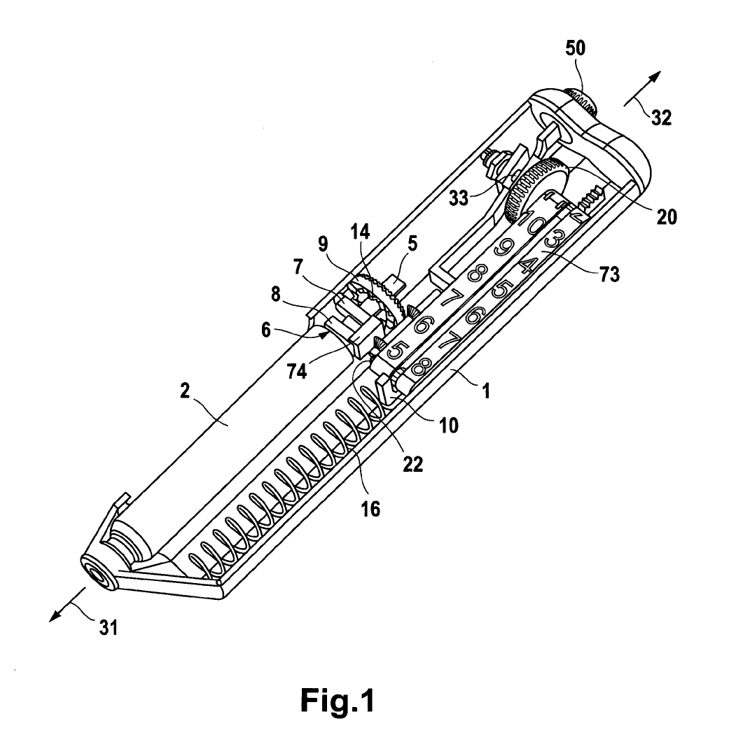

FIG. 1 shows a three-dimensional view of an embodiment of a drug delivery device.

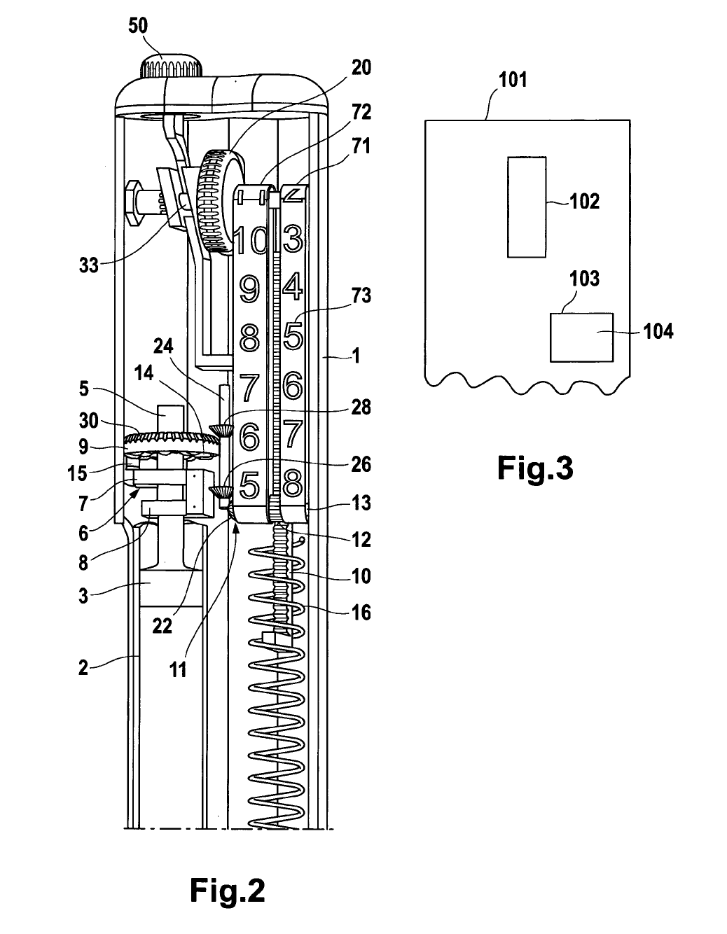

FIG. 2 shows a front view of the drug delivery device.

FIG. 3 shows the proximal part of an upper part of a housing of the drug delivery device.

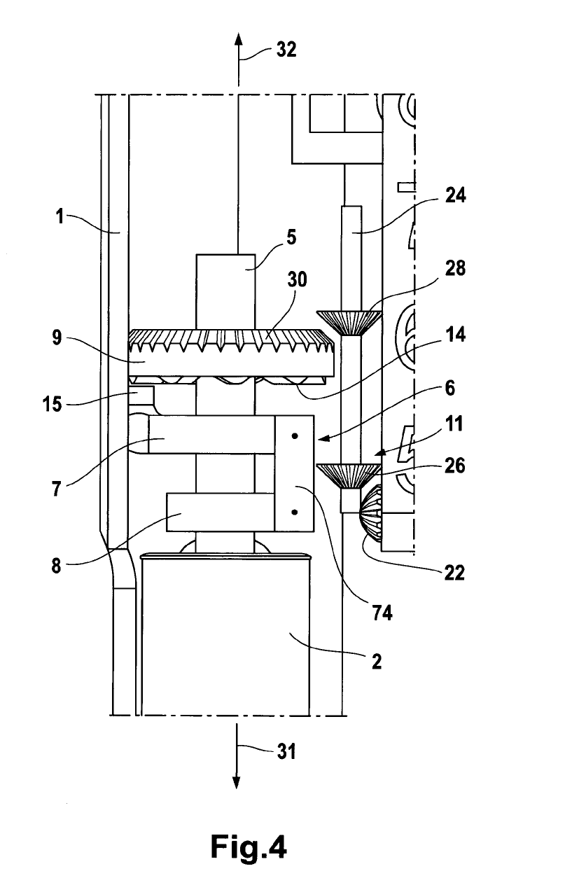

FIG. 4 shows a detailed view of a drive mechanism of the drug delivery device.

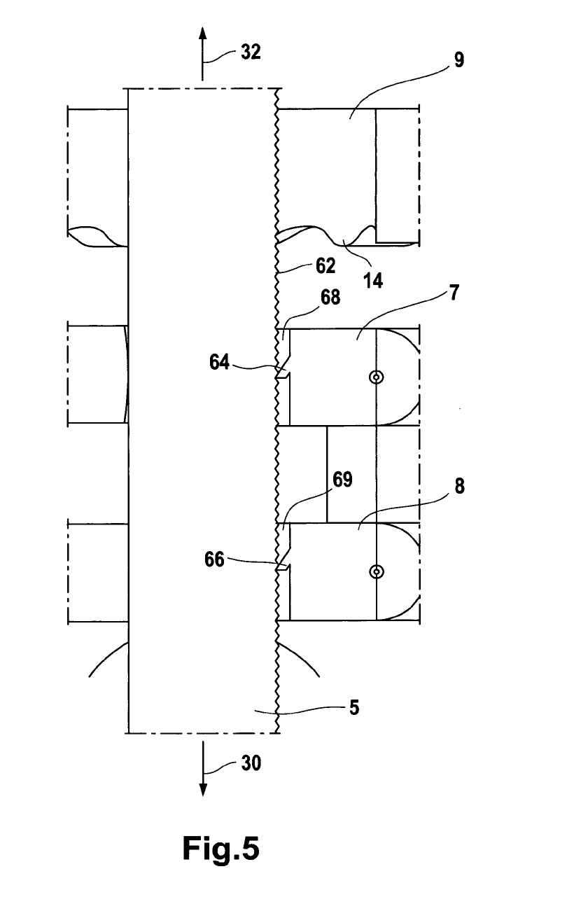

FIG. 5 shows details of a ratchet mechanism of the drug delivery device.

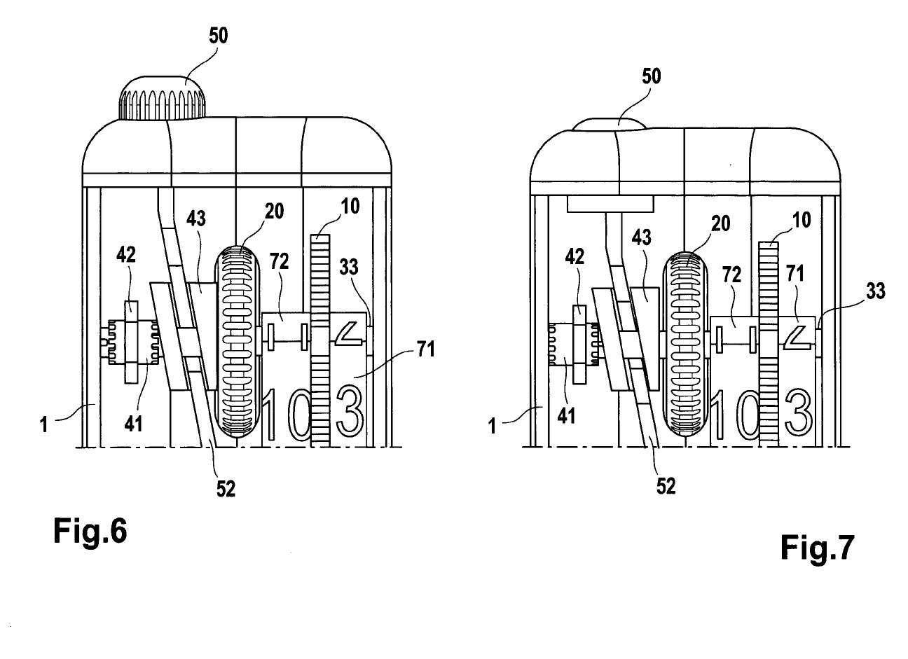

FIGS. 6 to 8 show a last dose indicator mechanism of the drug delivery device.

FIG. 1 shows a three-dimensional view of an embodiment of a drug delivery device, the upper part of the housing being removed for showing the inside of the drug delivery device including a drive mechanism. FIG. 2 shows a front view of the drug delivery device shown in FIG. 1, the upper part of the housing being removed and the distal part of the device not being shown. Some components of the drug delivery device are shown in FIG. 2 in more detail. FIG. 1 and FIG. 2 are described in connection.

The drug delivery device has a distal end and a proximal end. The term “distal end” of the drug delivery device or a component thereof may refer to that end of the device or the component which is closest to the dispensing end of the device. The term “proximal end” of the drug delivery device or a component thereof may refer to that end of the device or the component which is furthest away from the dispensing end of the device. The distal direction is indicated by an arrow 31. The proximal direction is indicated by an arrow 32.

The drug delivery device comprises a housing 1. The term “housing” shall preferably mean any exterior housing (“main housing”, “body”, “shell”) or interior housing (“insert”, “inner body”) which may have a unidirectional axial coupling to prevent proximal movement of specific components. The housing may be designed to facilitate the safe, correct and comfortable handling of the medication delivery device or any of its mechanisms. Usually, it is designed to house, fix, protect, guide, and/or engage with any of the inner components of the delivery device (e.g. the drive mechanism, cartridge, piston, piston rod, lead screw), preferably by limiting the exposure to contaminants, such as liquid, dust, dirt etc. In general, the housing may be a unitary or a multipart component of tubular or non-tubular shape. In this embodiment the housing 1 is non-axisymmetric.

A cartridge 2 is located inside the housing 1. The cartridge 2 containing a liquid drug or medicament has a distal end covered by a membrane that may be punctured by a needle (not shown) for drug delivery. A bung 3 (not shown in FIG. 1 but in FIG. 2) is located at the proximal end of the cartridge 2, the bung 3 being moveable distally along the inner side wall of the cartridge 2, thereby reducing the volume of the drug containing chamber of the cartridge 2 so that the drug is ejected through the needle (not shown). The cartridge 2 is held in its position by parts of the interior housing in such a manner that the distal end of the cartridge 2 is located at a distal opening of the housing 1, which allows attachment of the needle to the cartridge 2 or the housing 1. The drug delivery device may be intended to accept a 1.5 ml cartridge 2 or a 3.0 ml cartridge 2, but the design could be adapted to accept other drug container sizes or formats. A detachable cap (not shown) may be provided for protection of the distal part of the drug delivery device.

A drive mechanism is located inside the housing 1, the drive mechanism being suitable for moving the bung 3 in the distal direction, thereby delivering the drug.

The drive mechanism comprises a piston rod 5 having a distal end which abuts the bung 3. The piston rod 5 is moveable in the distal direction with respect to the housing 1 and the cartridge 2, thereby pushing the bung 3 distally, which causes drug delivery. The piston rod 5 may have a rectangular or circular cross section. In this embodiment the piston rod 5 is rather flat and has a rectangular cross section.

A follower member 6 which is suitable for driving the piston rod 5 is coupled with the piston rod 5 in such a manner that a distal movement of the piston rod 5 with respect to the follower member 6 is allowed while preventing a motion in the proximal direction. The follower member 6 comprises a first arm 7 and a second arm 8, that are connected with a holding section 74, extending angular, in particular orthogonal, with respect to the piston rod 5. The position of the follower member 6 with respect to the housing 1 is fixed, e.g. by connecting the housing 1 and the holding section 74. Nevertheless the first arm 7 is moveable with respect to the piston rod 5, thereby serving as a lever.

A rotatable cam member 9 drives the piston rod 5 by means of the follower member 6. The disk-shaped cam member 9 has a face cam that comprises a plurality of protrusions 14 arranged in a circle, the protrusions 14 extending towards the first arm 7. A cam gear 30 is provided on the opposite face of the cam member 9, the cam gear 30 being formed as bevel gear, the tooth-bearing face of the gear being conically shaped.

A contact section 15 of the first arm 7 abuts the cam face in such a manner that the rotating cam member 9 pushes the first arm 7 distally when it slides over a protrusion 14. The first arm 7 moves proximally when it moves along the region between the protrusions 14, thereby causing a reciprocating movement of the first arm 7.

The first arm 7 is coupled to piston rod 5 by a ratchet mechanism (clearly shown in FIG. 5) in such a manner that the reciprocating movement of the first arm 7 drives the piston rod 5 in the distal direction. The ratchet mechanism will be described in connection with FIGS. 4 and 5.

Returning to FIGS. 1 and 2, the drug delivery device further comprises a spring element 16 located next to the cartridge 2 inside the housing 1. The spring element 16 is deformed during the dose setting state of the drug delivery device in such a manner that it stores mechanical energy and at least partly relaxes during the delivery state of the delivery device, thereby exerting a spring force on some of the components. This spring-loaded drive mechanism may be similar to the design of an auto-injector, which, however, can be used only once in contrast to the multi-dose drug delivery device as described.

The spring element 16 is releasably coupled to the cam member 9 in such a manner that the elastic force of the relaxing spring element 16 drives the cam member 9 during the delivery state. Since the cam member 9 and the piston rod 5 are driven by the spring force, such a drive mechanism allows an easy way of drug delivery, where the spring-loaded mechanism may be triggered by a simple switch mechanism.

In this embodiment, the spring element 16 is formed as a helical compression spring, which is compressed during the dose setting state. The distal end of the spring element 16 may abut the housing 1, which allows compression of the spring element 16 by pushing the proximal end of the spring element 16 into the distal direction.

The spring element 16 is connected with a toothed rack member 10, which comprises a multitude of teeth arranged parallel with respect to the axis of the rack member 10. The longitudinal axes of the spring element 16 and the rack member 10 run in a same direction. The rack member 10 may have holes arranged along the rack member's axis, the proximal windings of the spring element 9 running through these holes thereby forming a connection between the spring element 16 and the rack member 10. Alternatively, the rack member 10 and the spring element 16 may be connected in another suitable way. There may be no mechanical connection but the movement of one component abutting the other one is transferred when one component pushes the other one. The spring element 16 may be compressed by a distal movement of the rack member 10 with respect to the housing 1, the distal end of the rack member 10 abutting the spring element 16. When the spring element 16 relaxes, the rack member 10 is moved in the proximal direction by the spring element 16 abutting the distal end of the rack member 10.

A gearbox 11 is coupled between the rack member 10 and the cam member 9, the gearbox 11 being suitable for transferring the linear movement of the rack member 10 in the proximal direction into a rotation of the cam member 9 during the drug delivery state.

The gearbox 11 may be decoupled from the cam member 9 in the dose setting state. No movement is transferred to the cam member 9 during the dose setting state, when the rack member 10 moves distally.

The gearbox 11 comprises a pinion 12, i.e. a toothed wheel or cylinder. The teeth of the pinion 12 may engage with the teeth of the rack member 10. The rack member 10 and the pinion 12 form a rack and pinion system converting the linear motion of the rack member 10 to a rotational motion of the pinion 12. Such a rack and pinion system comprises a circular gear, i.e. the pinion 12, engaging teeth on a linear gear bar, i.e. the rack member 10; in such a manner that rotational motion applied to the pinion 12 causes the rack member 10 to move, thereby translating the rotational motion of the pinion 12 into the linear motion of the rack member 10. Vice versa the linear motion of the rack member 10 may be translated into a rotation of the pinion 12.

The pinion 12 is connected via a first shaft 13 with a first gear 22 that is formed as a bevel gear located on a face side of the first shaft 13, the first gear 22 having a tooth-bearing face and being conically shaped. The first shaft 13 is arranged orthogonal to the rack member 10. It allows transferring the rotational movement of the pinion 12 to the first gear 22. The pinion 12, the first shaft 13 and the first gear 22 may be integrally formed.

The gearbox 11 further comprises a second shaft 24 that is arranged parallel to the rack member 10. The second shaft 24 is arranged angular, e.g. orthogonal, with respect to the first shaft 13. The second shaft 24 comprises a second gear 26 and a third gear 28, each being a bevels gear and having a tooth-bearing face and being conically shaped. The second shaft 24 is moveable with respect to the first gear 22 and the cam member 9 in such a manner that in the delivery state the second gear 26 engages with the first gear 22 and the third gear 28 engages with the cam gear 30. In the dose setting state of the drug delivery device the second gear 26 does not engage with the first gear 22 and the third gear 28 does not engage with the cam gear 30. Gears 22, 26, 28, 30 may alternatively be spur gears. FIG. 4 shows the arrangement of the gears 22, 26, 28 and the cam member 9 in detail.

When the first and second gears 22, 26 are engaged, the rotation of the first shaft 13 is transferred to the second shaft 24. Since the third gear 28 also engages with the cam gear 30 the rotational movement of the second shaft 24 is transferred to the cam member 9, thereby rotating it.

Returning to FIGS. 1 and 2, the rack member 10 may be moved by means of a scroll wheel 20 that serves as dosage selector. The scroll wheel 20 is located in the proximal region of the drug delivery device. It is mounted in such a manner that a sector of the scroll wheel 20 protrudes out of the housing 1, which allows manually rotating the scroll wheel 20 by the user of the drug delivery device. The scroll wheel 20 may be thumb operable.

The scroll wheel 20 is coupled to a third shaft 33 which is connected with the housing 1 in such a manner that the third shaft 33 and the scroll wheel 20 may rotate. The scroll wheel 20 may be an integral part of the third shaft 33. When setting a dose, the rotation of the scroll wheel 20 causes compression of the spring element 16 via a belt gear.

The third shaft 33 and the first shaft 13 are coupled via a first belt 71. A belt is a loop of flexible material used to link shafts mechanically. The first belt 71 runs over wheel means on the first shaft 13 and wheel means on the third shaft 33, thereby forming the belt gear that transmits rotational movement from one of the wheel means to the other of the wheel means. A wheel means may be a region of the shaft or a wheel-shaped means on the shaft the belt runs over. The wheel means does not necessarily protrude from the shaft.

The first belt 71 transmits the rotational movement of the third shaft 33, which is driven by the scroll wheel 20, to the first shaft 13, thereby moving the rack member 10 in dependence on the rotation of the scroll wheel 20.

The first belt 71 may be embodied as a toothed belt having a plurality of teeth on the inner face that may engage with teeth on the outer faces of the shafts forming the wheel means. Alternatively the first belt 71 may be embodied as a flat belt having a rectangular, trapezoid or ellipsoid cross-section without teeth.

In one embodiment the first belt 71, that is a flat belt, runs over pulleys being wheel means on the first and third shafts 13, 33; the pulleys being designed to support movement of the belt 71 along their circumferences. One embodiment of a pulley may have a groove between two flanges around its circumference, the belt running over the pulley inside the groove. The pulley may be fixed to the shaft or may be an integral part of the shaft, which allows direct transfer of the shaft movement onto the belt.

In an alternative embodiment the first belt 71, having teeth, runs over toothed wheels on the first and third shafts 13, 33 that are designed to support the movement of the belt 71 along their circumferences. The teeth of the toothed wheels may be arranged between two flanges around its circumference, the belt being guided by the flanges. Alternatively the shafts may have teeth whose widths extend along the whole length of the shaft. In such an embodiment of the shafts a region of one tooth engages with first belt 71 while the adjacent region of the same tooth engages with the rack member 10. The toothed wheel may be fixed to the shaft or may be an integral part of the shaft, which allows direct transfer of the shaft movement onto the belt.

The pulleys and toothed wheels are examples of wheel means over which the first belt 71 is looped.

The first belt 71 comprises marks 73 arranged along the loop. The marks are indications for the set dose of the state of the drug delivery device. Such marks may include signs or symbols that may represent states of the device, e.g. the amount of the set dose. The marks may be printed. Alternatively they are structures on the surface of the belt.

In one embodiment the marks may include pictograms indicating a full or empty cartridge. In one embodiment the digits 0 to 9 are placed equidistantly along the loop. The digits may indicate the ones or units of the dose amount that is set.

A second belt 72 is arranged parallel to the first belt 71; the second belt 72 running over wheel means which may be formed, as mentioned above, between the first and third shafts 13, 33. The second belt 72 also comprises marks placed along the loop. In this embodiment a blank and the numbers 1 to 12 are arranged on the second belt 72. These symbols are arranged equidistantly. Alternatively the digits 0 to 9 may be arranged on the second belt 72.

The belts 71, 72 serving as an indicator do not need to indicate using numbers: the indicator could include, but not be limited to, one or more of the following, perhaps in combination: text; icons, symbols or images; colour; Braille or other tactile surface.

The first and second belts 71, 72 serve as the dose indicator, wherein parts of the belts 71, 72 are visible in a window in the housing 1. FIG. 3 shows the proximal part of the upper part 101 of the housing 1 having a first opening 102 through which the scroll wheel 20 protrudes and a second opening serving as window 103 for the number belt dose indicator (in one embodiment comprising first belt 71 and second belt 72).

It may be desired in some applications to cover the window 103 in the housing to prevent debris from entering the opening and either looking unsightly or interfering with the operation of the dose indicator mechanism. Means to prevent this include covering the opening with a transparent label. One embodiment, e.g. an insulin pen injector, requires a label for regulatory reasons and therefore no additional parts are necessary. In one embodiment the window 103 is covered by a transparent material 104. Further information, e.g. information about the drug, may be provided on the cover 104. Suitable materials for the window cover 104 may include thermoplastic injection moulding or glass. In either material, the cover 104 could be shaped to affect a lens and therefore increase the apparent size of text, numbers or other information on the belts 71, 72. Such a cover 104 may have a bulb serving as enlarging lens.

The digits arranged on the visible parts of the belts 71, 72 indicate the set dose. The visible parts are the parts that are visible through the window 103. The digit on the visible part of the first belt 71 indicates the units of the amount of the set dose. The digit on the visible part of the second belt 72 indicates the tens of units of the amount. Both parts in combination indicate the amount of the dose set. When no dose is set a blank on the second belt 72 and “0” on the first belt 71 indicates this state. When the dose is increased by rotating the scroll wheel 20, the third shaft 33 also rotates; thereby moving the first belt 71 and the units on the first belt 71 increase until “9” is shown. When the “0” on the first belt becomes visible again, the second belt 72 moves so that the next tens digit is visible, this symbol remaining visible until the first belt 71 shows “0” again. Then the next tens digit becomes visible on the second belt 72. During the transition from “9” to “0” on the first belt 71, the first belt 71 indexes the second belt 72 showing the tens, using some type of escapement mechanism. Such an escapement mechanism couples the first and the second belts 71, 72 in such a manner that the second belt 72 moves by one increment, i.e. from one digit to the following digit, after one revolution of the first belt 71.

A first wheel means over which the first belt 71 runs is fixed to the third shaft 33. A second wheel means over which the first belt 71 runs is fixed to the first shaft 13. A third wheel means over which the second belt 72 runs is coupled to the third shaft 33. A fourth wheel means over which the second belt 72 runs is coupled to the first shaft 13. In one embodiment the first and second wheel means are connected with the third and first shafts 33, 13, respectively. The first wheel means may be coupled with the third wheel means over which the second belt 72 runs in such a manner that the completion of one revolution of the first belt 71 moves the second belt 72 one increment. Such coupling may be formed by a protrusion on the first disk-shaped wheel means or on the first belt 71 engaging with the third disk-shaped wheel means in such a manner that, after the completion of one revolution of the first belt 71, the third wheel means moves the second belt 72 by one increment. The first belt 71 may have a finger which engages with the third wheel means when the belt section with the finger runs over the first wheel means, thereby stepwise moving the third wheel means and the second belt 72 after one revolution of the first belt 71. The third wheel means serves as escapement means which merely performs a stepwise rotation. The belts 71, 72 may be driven by alternative mechanisms that may be based on the functional principle of mechanical counter and stepping gears, e.g. roller counters, where the wheels of the mechanical counter mechanism serve as wheel means for guiding and driving the belts.

The first and second belts 71, 72 may be coupled to the first shaft by a mechanism as described above.

Though only two belts have been described, more than two belts may be used, where the belts are driven by a mechanical counter mechanism having more than two wheel means.

The flexible nature of the belts 71, 72 allows the dose indicator mechanism to be arranged in a wider range of form factors. In many cases this will allow a more efficient use of space within the device packaging.

Additionally the dose indication on the belts 71, 72 is presented on a much flatter surface than a conventional indication on a cylinder or a sleeve. This allows larger perceived dose numbers and less distortion when viewing at an angle. Should the device require additional magnification, a flatter display will allow greater freedom with the lens design and potentially less optical distortion.

One embodiment of the drug delivery device (not shown) may comprise a dose indicator variant. Depending on the size of the maximum dose and the incremental display of individual units a spool based mechanism could be considered. In this variant the belt or tape loop mechanism would be replaced with a spool to spool system. This would allow the dose to be dialled up or down as usual but the dosage range would be limited by the length of the spool as opposed to a looping tape or belt.

The drug delivery device further comprises a last dose indicator mechanism comprising a last dose nut 42 and a last dose slider 43 that is coupled with the third shaft 33. The last dose indicator mechanism will be described later in connection with the FIGS. 6 to 8.

Returning to FIGS. 1 and 2, the drug delivery device further comprises a button member 50 protruding out of the housing 1 and serving for initiating the drug delivery. The button member 50 is moved distally when the user pushes it. When the button member 50 is released the button member 50 moves proximally, e.g. by means of a spring (not shown).

The button member 50 is connected with a switch rod 52 that is coupled with the second shaft 24 in such a manner that pushing the button member 50 causes movement of the second shaft 24 in the distal direction, thereby coupling the gearbox 11 and the cam member 9 since the first and the second gears 22, 26 as well as the third gear 28, and the cam gear 30 engage.

FIG. 4 shows the cam member 9 driving the piston rod 5 by means of the follower member 6 in detail. FIG. 5 shows details of the ratchet mechanism formed by a toothed rack 62 and a driving pawl 64. The piston rod 5 comprises the toothed rack 62 that may be an integral part of the piston rod 5. The toothed rack 62 comprises a plurality of teeth that are uniform but asymmetrical, with each tooth having a moderate slope on one edge and a much steeper slope on the other edge. The proximal faces of teeth have steeper slope than the distal faces of the teeth.

The follower member 6 comprises the holding section 74 which is located parallel with to the piston rod 5; the first and second arms 7, 8 extending substantially orthogonal with respect to the holding section 74. The holding section 74 is in fixed connection with the housing 1 in such a manner that no movement is possible with respect to the housing 1. The holding section 74 may be integral to the housing 1.

The first arm 7 servers as a lever pivoted at the holding section 74. The first arm 7 and the holding section 74 may be separate parts connected by a hinge. Alternatively they are integrally formed, the first arm 7 being movable due to the elasticity of the material.

The first arm 7 includes the driving pawl 64 coupled to the toothed rack 62 in such a manner that the reciprocating movement of the driving pawl 64 drives the piston rod 5. The first arm 7 may have a trench 68 through which the piston rod 5 runs. The driving pawl 64 is located on the side wall of the trench 68 that is located near the holding section 74. The other side wall of the trench 68 has a concave shape which allows an oscillating movement of the first arm 7 with respect to the piston rod 5.

When the driving pawl 64 is moved proximally with respect to the rack 62, the driving pawl 64 easily slides up and over the gently sloped edges of the teeth of the rack 62. When the driving pawl 64 is move in the opposite direction, i.e. the distal direction, however, the driving 62 pawl will catch against the steeply sloped edge of the first tooth it encounters, thereby locking it against the tooth and preventing distal movement of the driving pawl 64 with respect to the piston rod 5. Thus the distal movement of the driving pawl 64 also moves the piston rod 5 distally.

The second arm 8 comprises a backlash pawl 66. The second arm 8 may be integral to the holding section 74 and/or the housing 1. The second arm 8 may have a trench 69 through which the piston rod 5 runs. The backlash pawl 66 is located on the side wall of the trench 69 that is located near the holding section 74. Alternatively, the backlash pawl may be in another position and interact with a different set of teeth on rack 62. The other side wall of the trench 69 runs parallel with and adjacent to the piston rod 5, which prevents deflection of the piston rod 5 with respect to the second arm 8. The second arm 8 does not drive the piston rod 5 but stays in its position. The second arm 8 serves for holding the piston rod 5 in its position when the driving pawl 64 slides proximally along the teeth. When the rack 62 is moved distally with respect to the second arm 8, the backlash pawl 66 easily slides up and over the gently sloped edges of the teeth. When the teeth would move in the opposite direction, e.g. when the first arm 7 moves proximally, however, the backlash pawl 66 will catch against the steeply sloped edge of the first tooth it encounters, thereby locking it against the tooth and preventing any motion in that direction. Thus the backlash pawl 66 prevents proximal movement of the piston rod 5 when the driving pawl 64 moves proximally along the rack 62.

The cam member 9 causes oscillation of the first arm 7 as described above, which causes the piston rod 5 to move axially by the reciprocation of the first arm 7 and the driving pawl 64. The piston rod 5 cannot retract because the backlash pawl 66 prevents it. Therefore, each oscillation of the driving pawl 64 acts to advance the piston rod 5.

The first arm 7 with the driving pawl 64 is a lever. The lever ratio converts the large displacement (with relatively low force) of the contact section 15 and the face cam protrusions 14 into a small displacement (with relatively high force) of the driving pawl 64 impacting to the piston rod 5. In other words, the lever arm ratio of the driving pawl 64 translates a large displacement on the cam member 9 to a small displacement at the driving pawl 64. In one embodiment one oscillation which moves the piston rod may be moved by one tooth. The displacement ratio may be useful to achieve good dose accuracy. This could be useful in meeting the challenge of the smaller dose volumes demanded by the high-concentration, and therefore low volume, drug formulations.

Moreover the use of the cam member 9 to drive the piston rod 5 may be a more compact and accurate way of translating rotational motion into axial motion than using a leadscrew.

The drug delivery device further comprises a last dose indicator mechanism shown in FIGS. 6 to 8 which illustrate different states of operation.

FIG. 6 shows the proximal part of the drug delivery device including the scroll wheel 20 connected with the third shaft 33 in such a manner that the third shaft 33 and the scroll wheel 20 may rotate with respect to the housing 1.

A last dose indicator mechanism comprises a last dose slider 41 that is formed as a sleeve having axially protruding teeth on its end faces. The last dose slider 41 is axially moveable along the third shaft 33 between a first position and a second position. In the first position the last dose slider 41 is splined to the third shaft 33 when the teeth of the last dose slider 41 engage with protrusion on the third shaft 33. In other words, in this position the last dose slider 41 is not rotatably moveable with respect to the third shaft 33. In the second position the last dose slider 41 is splined with the housing 1, e.g. when the teeth on the other side of the last dose slider 41 engage with a protrusion on the inner wall of the housing 1. In this position the last dose slider 41 is rotatably moveable with respect to the third shaft 33. A last dose nut 42 is arranged on the last dose slider 41, the components being coupled in such a manner that the last dose nut 42 axially moves along the last dose slider 41, when the latter rotates. In one embodiment the last dose nut 42 may have an asymmetrical, e.g. rectangular, contour, which prevents its rotational movement with respect to the housing 1. The last dose nut 42 and the last dose slider 41 may be coupled by a threaded connection, thereby transferring the rotational movement of the last dose slider 41 into an axial movement of the last dose nut 42. The last dose nut 42 is coupled via the last dose slider 41 and the third shaft 33 with the scroll wheel 20. Increasing the set dose by rotating the scroll wheel 20 causes the last dose nut 42 to move along the last dose slider 41 away from the scroll wheel 20 towards the housing 1. Decreasing the set dose by scrolling into the opposite direction moves the last dose nut 42 back along the last dose slider 41 and away from the housing 1.

A sleeve member or cam 43 is located between the scroll wheel 20 and the last dose slider 41. The third shaft 33 may rotate with respect to the sleeve member 43. The sleeve member 43 has an angular side facing the last dose slider 41. Alternatively the sleeve member 43 may be symmetrically formed. The other side may be coupled with the scroll wheel 20 by ratchet means (not shown). Such ratchet features on the sleeve member 43 prevent the scroll wheel 20 from unwinding under the force from the spring element 16, the force impacting via the rack member 10, the pinion 12 and the belts 71, 72.

The sleeve member 43 has an angularly running trench or hole running through the middle section of the switch rod 52, thereby preventing rotational movement of the sleeve member 43 with respect to the housing 1. Nevertheless the middle section is moveable along the trench or the hole.

The button member 50 is coupled by the switch rod 52 with the sleeve member 43 in such a manner that the distal movement of the button member 50 moves the sleeve member 43 towards the housing 1, thereby decoupling the splined connection between the last dose slider 41 and the third shaft 33 and moving the last dose slider 41 towards the housing 1 into the splined connection with the housing 1. When the switch rod 52 is moved in the distal direction, the angular middle section moves the sleeve member 43 towards the last dose indicator mechanism, thereby pushing the last dose slider 41 towards the housing 1.

FIG. 7 shows the last dose indicator mechanism in the delivery state when the last dose slider 41 is in splined connection with the housing 1 after pushing the button member 50. In this state the force of the relaxing spring element 16 rotates the first shaft 13 and, via the belt gear, the third shaft 33. When the third shaft 33 rotates as the dose is delivered, the last dose nut 42 does not move back to its zero position with respect to the last dose slider 41: it remains in its position, thereby “remembering” the delivered dose volume.

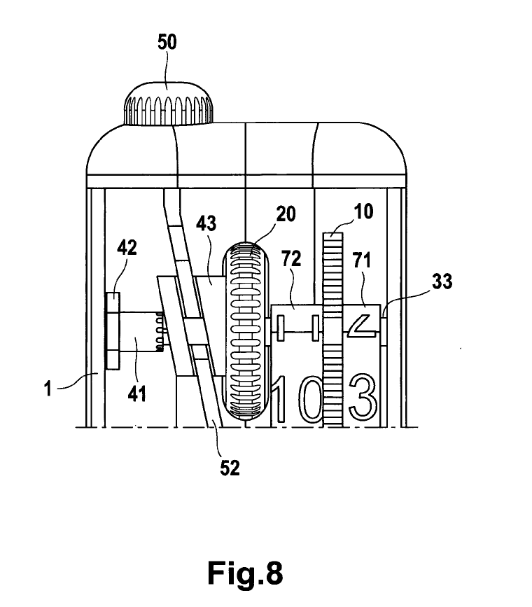

As the drug in the cartridge 2 is used up, the last dose nut 42 moves progressively along the last dose slider 41 towards the inner surface of the housing 1. If the user tries to set a dose greater than the volume remaining in the cartridge 2, the last dose slider 41 rotates as per usual but the last dose nut 42 contacts the inner surface of the housing 1 and prevents the set dose from increasing. FIG. 8 shows this state. The last dose nut 42 abuts the inner wall of the housing 1 which prevents further rotation of the last dose slider 41 and the third shaft 33 that are in splined connection. The movement of the scroll wheel 20 is stopped.

The operation of the drug delivery is performed as follows. During the first dose setting and delivery the intended user steps are: The cap (not shown) is removed. A needle (not shown) is fit onto the distal end of the drug delivery device. The needle may be a standard double-ended needle described in ISO 11608-2:2012. A priming dose, e.g. 2 IU of insulin formulation, is dialled by rotating the scroll wheel 20 which serves as a dosage selector. The “priming” dose is dispensed by pressing the button member 50 on the proximal end of the drug delivery device. The required dose for injection into the body is dialled by rotating the scroll wheel 20. The needle is inserted into the skin. The dialled dose is dispensed by pressing the button member 50. The needle is removed from the skin. The needle is removed from the drug delivery device and the cap is replaced.

Priming is the act of preparing the drug delivery device for first use. In pen injectors this may mean setting and delivering one or more small doses into air so that the play (any clearances) and tolerances in the device are removed and that components are placed into suitable compression or tension. Safety shots are where the user sets and delivers one or more small doses into air before each injection to ensure that the needle is not blocked. The clamshell design of the casework may allow the components to be positioned during assembly for minimum clearance and therefore minimum priming volume. For both priming the device and for safety shots, the user will set a small dose and inject that dose into air and repeat until a drop of medicament is observed at the tip of the needle.

The user sets a dose by rotating the scroll wheel 20. This motion is transferred by the third shaft 33 to the belt gear. Since the belts 71, 72 move, the digits on the belts 71, 72, that are visible in the window 103, change. The user moves the scroll wheel 20 as long as the desired amount of the dose is shown in the window 103. In other words, rotating the scroll wheel 20 indexes the belt transmission, which drives via the first shaft 13 the pinion 12, thereby moving the rack member 10 distally; this causes the compression of the spring element 16. Since the gear box 11 is decoupled from the cam member 9 in the dose setting state, the cam member 9 and the piston rod 5 do not move.

Drug delivery is achieved by pressing the button member 50. When the button member 50 is pressed it has a small (˜1 mm) travel in which no action takes place. This feature prevents accidentally drug delivery. The button member 50 will tend to return to its original position due to force from a spring (not shown).

When pushing the button member 50 the switch rod 52 is moved distally. In response to this movement several actions take place. The switch rod 52 pushes the sleeve member 43 so that the last dose slider 41 is splined no longer to the third shaft 33 but is instead splined to the housing 1. The ratchet feature on the sleeve member 43 releases the scroll wheel 20, allowing the spring element 16 to drive the rack member 10 proximally, since the third shaft 33 may rotate. Moreover, the switch rod 52 pushes the second shaft 24 with the second and third gears 26, 28 axially in the distal direction so that the drive from the spring element 16 is connected via the gear box 11 and the cam member 9 to the ratchet mechanism on the piston rod 5 and the follower member 6.

When the rack member 10 is driven back to the proximal direction by the spring element 16, the rack member 10 drives the first shaft 13, thereby driving the belts 71, 72 and in turn driving the gears 22, 26, 28, 30 and therefore the cam member 9. The cam member 9 makes the first arm 7 and the driving pawl 64 oscillate, which causes the piston rod 5 to move axially. The piston rod 5 cannot retract because the backlash pawl 66 prevents it. Therefore, each oscillation of the driving pawl 64 acts to advance the piston rod 5.

The drug delivery may be interrupted which allows to deliver merely a part of the set dose. If the axial pushing force on the button member 50 is removed, the button member 50 returns to its initial axial position and therefore the second gear 26 is disconnected from the first gear 22 and the third gear 28 is disconnected from the cam member 9, which stops driving the piston rod 5, thereby stopping drug delivery. Since the switching rod 52 moves proximally, the sleeve member 43 moves towards the scroll wheel 20. Thus the ratchet feature on the sleeve member 43 engages with the scroll wheel 20. Moreover, the last dose slider 41 is no longer splined to the housing 1 and is instead splined to the third shaft 33, which means that last dose protection will work, i.e. the last dose nut 42 moving towards the housing 1, if the user sets a greater dose than the volume remaining in the cartridge 2. The dose can be changed by rotating the scroll wheel 20 and pressing the button member 50 restarts the injection manoeuvre.

Nevertheless, the total dose may be injected by pushing the button member 50 until the dose is delivered. Delivery of the total dose is indicated by return of the belts 71, 72 to their initial positions. The end of injection can be indicated by a feature which makes one or more audible, visual and/or tactile signals, such as a “click” when two parts move relative to one another near or at the end of injection.

After drug delivery it may be beneficial that the needle is not removed from the skin immediately but stays there for a hold time. The “hold time” is the period from when the mechanism has stopped moving, most typically indicated by the dose indicator returning to its initial position, to when the dose is fully delivered and the user can remove the needle from the patient without affecting the delivered dose volume.

Hold time is required on some devices because, if the user injects the drug too fast, it can take some time, typically a few seconds, for the elasticity of the mechanics to equilibrate and deliver the correct volume; and for the drug formulation to disperse in the tissue and reduce the back pressure.

In this embodiment the drug delivery device is entirely driven by the spring element 16 and therefore the injection speed should be much less variable than for a manual device. If the maximum injection speed is consistent and minimised, then the hold time could be reduced.

The drive mechanism including a linear ratchet concept offers greater freedom to develop non-axis-symmetric devices contrary to injector pens following an axis-symmetric form factor which is largely dictated by the injection mechanism and also the helical number sleeve.

Non-axis-symmetric devices offer improved usability for handling and grip, and clearer, easier to read dose numbers. There is also an increased device differentiation since many conventional pen injectors are indistinguishable from one another, which could be a problem for users in identifying the correct device to use for a given use case, for example, selecting between long-acting and short-acting insulin).

The device is designed to be disposable (in that the cartridge 2 cannot be replaced by the user or healthcare professional) but a reusable variant of the device could be created by making the cartridge holder removable and allowing the resetting of the piston rod 5.

When the device is at rest the compressed spring element 16 has enough preload such that if the user selects the minimum dose the device will be able to deliver that minimum dose. At rest the dose indicator displays “0” or the equivalent marking to show that no dose has been selected.

The invention will be used to inject a liquid drug such as insulin. This may be for human use. Nevertheless the invention is not limited to such embodiments.

The most relevant applications are in dispensing mechanisms, a few examples being given in the following. The mechanism may be used for drug delivery devices such as pen injectors or autoinjectors. It can be also used for medical devices such as dispensers of antiseptic creams, analgesic creams, detergents and so on. It can be used for devices for dispensing adhesives, lubricants, paints, detergents and suchlike. These could be used in professional applications such as engineering workshops or in consumer applications such as “do it yourself” products or “fast moving consumer goods”. It can be used for food dispensers for non-rigid foods such as tomato sauce, crushed garlic, cheese, butter, juice, smoothie, soup, coffee, tea, jam, peanut butter and so on. It can be used as positioning mechanisms such as the stages used on milling machines, lathes, visual coordinate measuring machines and optics equipment.

The features of the embodiments mentioned above may be combined. The layout, function, and number of components may be changed in other embodiments.

### REFERENCE NUMERALS

1 housing
2 cartridge
3 bung
5 piston rod
6 follower member
7 first arm
8 second arm
9 cam member
10 rack member
11 gear box
12 pinion
13 first shaft
14 protrusions
15 contact section
16 spring element
20 scrolling wheel
22 first gear
24 second shaft
26 second gear
28 third gear
30 cam gear
31 arrow
32 arrow
33 third shaft
41 last dose slider
42 last dose nut
43 sleeve member
50 button member
52 switch rod
62 rack
64 driving pawl
66 backlash pawl
68 trench
69 trench
71 first belt
72 second belt
73 mark
74 holding section
101 upper housing
102 opening
103 window
104 cover

1. An indicator for a delivery device comprising a belt (71) having marks (73) for dose indication and/or status indication.

2. The indicator according to claim 1,
wherein the belt (71) runs over a first wheel means and a second wheel means; the belt (71), the first and second wheel means forming a belt gear suitable for transferring rotation from one of the wheel means to the other wheel means.

3. The indicator according to claim 1 or 2,
wherein the belt (71) comprises teeth on its inner side, the teeth engaging with teeth on at least one of the first and second wheel means.

4. The indicator according to any of the previous claims,
wherein at least one of the first and second wheel means comprises a pulley.

5. The indicator according to any of the previous claims,
further comprising a further belt (72) having marks for dose indication and/or status indication.

6. The indicator according to claim 5, wherein the belt (71) and the further belt (72) are arranged adjacent and substantially in parallel to each other, the marks (73) on the belt (71) including digits that indicate units of an amount of a set dose and the marks (73) on the further belt (72) including digits that indicate tens of the amount of the set dose.

7. The indicator according to claim 5 or 6,
wherein the belt (71) is coupled with the further belt (72) in such a manner that the further belt (72) is moved one increment when the belt (71) completes one revolution.

8. The indicator according to claim 7,
further comprising an escapement mechanism.

9. A delivery device comprising the indicator according to any of the claims 2 to 8 and further comprising a moveable dosage selector (20) coupled by a shaft (33) with the first wheel means in such a manner that a movement of the dosage selector (20) causes rotation of the first wheel means.

10. The delivery device according to claim 9, wherein the second wheel means is coupled with a mechanism (13, 11, 9, 6, 10, 12, 16) for driving a piston rod (5), the mechanism (13, 11, 9, 6, 10, 12, 16) having at least one component (13, 12) that is moved by the second wheel means.

11. The delivery device according to claim 9 or 10 or comprising the indicator to claims 1 to 8, further comprising a housing (1) having a window (103), a part of the belt (71) being visible through the window (103), wherein the visible mark (73) indicates the set dose or the status of the device.

12. The delivery device according to claim 11 wherein the window (103) is covered by a translucent or transparent material (104).

13. The delivery device according to claim 11 or 12 wherein the opening is covered by a lens.

14. The delivery device according to any of the claims 10 to 13 being a drug delivery device.
